(19) United States
(12) Patent Application Publication
(10) Pub. No.: US 2023/0181790 A1
(43) Pub. Date: Jun. 15, 2023

(54) RADIOACTIVE SHEAR THINNING BIOMATERIAL COMPOSITION AND METHODS FOR USE

(71) Applicant: Boston Scientific Scimed Inc., Maple Grove, MN (US)

(72) Inventors: Ehsan Jabbarzadeh, Columbia, SC (US); Sara Eslambolchi Moghadam, Santa Clara, CA (US); Karen Dubbin, Mountain View, CA (US)

(73) Assignee: Boston Scientific Scimed Inc., Maple Grove, MN (US)

(21) Appl. No.: 18/080,899

(22) Filed: Dec. 14, 2022

Related U.S. Application Data
(60) Provisional application No. 63/289,468, filed on Dec. 14, 2021.

(51) Int. Cl. A61L 24/00

(52) U.S. Cl. CPC A61L 24/001; A61L 2430/36

(57) ABSTRACT

The present invention is a method and device for treating solid tumors utilizing shear thinning biomaterials compositions containing beta- or alpha emitting radiation sources, polymer matrix, and/or radiopaque agent. The novel radioactive composition which is disclosed here, can be injected percutaneously or via transcatheter vascular route into the target environment for the locoregional treatment. This invention is comprised of a shear thinning biomaterial which, when combined with a radioactive isotope source, can provide a therapeutic dose of radiation locally to the tumor site minimizing the risk of damage to surrounding tissue. The device may be used either as the primary tumor treatment or for treatment of residual cancer cells after excision of the primary tumor. The present invention provides a method for making the shear thinning radioactive biomaterial composition, as well as a method for utilizing the composition as a part of the treatment method.

### CROSS REFERENCE TO RELATED APPLICATIONS

This application claims the benefit of U.S. Provisional Patent Application Ser. No. 63/289,468, filed on Dec. 14, 2021, the disclosure of which is incorporated herein by reference.

### FIELD OF THE INVENTION

The present disclosure relates to therapeutic radiology and cancer therapy. More particularly, the present disclosure is directed to a shear thinning biomaterial comprising radioactive materials.

### BACKGROUND

Radiotherapy has become one of the most prominent and effective modalities for cancer treatment, can be used alone or in combination with surgery, chemotherapy, and immunotherapy, and is the standard of care in approximately half of all cancer cases worldwide. Though different forms of radiotherapy exist, the treatment generally relies on the use of radioactive isotopes (also referred to herein as radioisotopes, radionuclides and radioactive agents) to serve as sources of ionizing radiation. Ionizing radiation, delivered to cancerous targets either from external or internal sources, causes damage to DNA that can induce apoptosis.

Depending on the type of cancer, radiotherapy can be used to treat localized cancer, either as palliative treatments to reduce symptoms, or to limit progression of the disease in incurable cases. Radiotherapy can also be used as an adjuvant therapy intra-operatively and post-operatively to help eliminate any residual tumor cells.

External beam radiotherapy is the most prevalent form of radiotherapy used in clinical settings and involves high-energy rays, in the form of photons (e.g., X-rays, gamma rays), protons, or particle radiation, from outside of the body to the specific tumor site. One major drawback of this therapy is the danger of damaging off-target, healthy tissues given the difficulty in directly targeting the cancerous tumors through external administration. Additionally, external beam radiation has a limited efficacy for larger tumors and is not amenable to deep tumors because the external radiation gives unwanted tissue absorption around the tumor area. As would then be expected, the use of external radiation requires careful attention as the patients should be placed in an exact position and the radiation dose should be precise to minimize irradiation and tissue damage to unintended area of the body. Even with this care, external irradiation can induce skin damage and can still result in unwanted tissue absorption around the tumor area.

In an effort to ameliorate these concerns, internal radiotherapy presents as a technique allowing for more localized dosing of therapeutic radiation to tumors using short range radionuclides placed within the body, usually adjacent to or directly into the tumor itself.

Brachytherapy is a type of internal radiotherapy that involves the placement of sealed radioactive sources adjacent to or within the cancerous tissue. The location in which the radioactive source is placed is used to classify the type of therapy, e.g., intracavitary brachytherapy, interstitial brachytherapy, intraluminal/intravascular brachytherapy or superficial brachytherapy. Brachytherapy can be further classified according to the dose rate applied, using the International Commission on Radiation Units stating that 0.4 to 2 Gray per hour (Gy·h⁻¹) is a low dose rate (LDR), 2 to 12 Gy·h⁻¹ is a medium dose rate (MDR), and a high dose rate (HDR) is regarded as being greater than 12 Gy·h⁻¹. HDR brachytherapy typically involves the temporary placement of a radioactive source, whilst LDR usually involves permanent implantation. In many instances, brachytherapy facilitates the delivery of a highly localized radiation dose that is unable to be achieved using conventional external beam radiation therapy.

Some brachytherapy devices and seeds are metal sealed radionuclides to provide for easier handling and delivery. One major drawback for this class of radioactive seeds is that the encapsulating metal absorbs a significant fraction of the low-energy beta and photon radiation emitted by the contained radionuclide. Thus, the current practice of brachytherapy based on the use of discrete encapsulated sources is limited. One issue with permanent brachytherapy seeds is that, in certain instances, they can require surgical implantation and removal. In other cases, the metal encapsulating material may remain permanently in the body and there is possibility of migration to the other parts of the tissue. A strategy to deliver radioactive seeds in a minimally invasive manner while preventing migration can improve implementation of this therapy.

Radioembolization is one method that has been explored for local, minimally invasive treatment of tumors without migration of the treatment vehicle. Radioembolization, generally, provides a minimally invasive form of internal radiotherapy that involves the delivery of radioactive microspheres as an embolic into the tumor vasculature to selectively irradiate tumors. The proximity of the microspheres to the tumor results in localized delivery of lethal doses of radiation to the tumor. Simultaneously, the microspheres cause a degree of embolization by occluding the blood vessels to prevent blood and nutrient flow to the tumor. Radioactive microspheres, however, while the most common mechanism of radioembolization, face disadvantages of their own. For one, radioembolization by microspheres requires vascular access to the tumor, which is not true in every case. Also, if vasculature is present into and out of the tumor, migration of the microspheres to other parts of the body, including reflux into nontargeted tissues and organs, is a possibility. Further, because the microspheres are delivered in aqueous media, the microspheres can settle and/or result in inhomogeneous delivery of radioactivity.

Thus, two major limitations of current radioembolization strategies are the potential for migration of radioactive microspheres away from the tumor site or for the spheres to settle in liquid dispersion, causing inhomogeneous irradiation. The present disclosure describes immobilization of radionuclides within a biomaterial. Whether used as an embolic or delivered percutaneously, radionuclides are immobilized due to the hydrogel mesh size preventing encapsulated particle release and interaction between radionuclides and silicate nanoparticles. Furthermore, the viscosity of the material prevents settling, increasing the homogeneity of radionuclides throughout the device.

In other words, embodiments of the present disclosure overcome limitations of current radioembolization strategies by incorporating radioactive sources into an injectable semi-solid biomaterial. The composition herein has shear thinning properties that allows it to be delivered to the tumor site via intravascular catheter or percutaneous injection but remain in place, proximate the tumor, upon extrusion in the tumor. Additionally, the biomaterial can serve as an embolic that enhances the anti-tumor effect of radiation by also limiting blood flow to the tumor. The polymer composition of the biomaterial is highly stable to radiation and incorporation of radioisotopes may augment current methods of radiation delivery.

### BRIEF SUMMARY

The present disclosure describes a method and device for treating solid tumors utilizing a shear thinning biomaterial composition comprising a beta- or alpha-emitting radiation source, a polymer matrix, and/or a radiopaque agent. The present disclosure is directed to a biomaterial composition comprising a radioisotope which is delivered via catheter to the vascular site or percutaneously injected to the tumor site. In an embodiment, the biomaterial composition is employed in a novel method to embolize blood vessel supplying blood to a solid tumor as well as to provide a therapeutic level of radiation to the blood vessel and/or tissue. In another embodiment, the biomaterial is percutaneously injected to the tissue to transfer the radioisotope.

The present disclosure relates to devices and methods for treatment of cancer, including liver cancer, kidney cancer, prostate cancer, brain cancer, and breast cancer. Other types of cancer may be treated using the methods and devices described herein, including lung cancer, bladder cancer, colon cancer, renal cancer, pancreatic cancer, thyroid cancer, glioblastoma, head and neck cancers and soft tissue sarcomas. More specifically the present disclosure relates in some embodiments to devices and methods for the treatment of solid tumors.

The present disclosure provides a variety of additional advantages. A high energy radiation source combined with shear thinning biomaterial with a preferred viscosity, delivers the radiation preferentially via catheter or percutaneous injection. Incorporation of radiation, through either a high energy beta or alpha emitter, will concentrate the zone of radiation exposure to the vicinity of tumor and reduce the radiation level and risk of damage to the healthy tissue. The slow resorption rate of the composition generously exceeds the half-life of the radionuclides of interest which advantageously reduces the risk of leaking to the other organs and tissues. The slow degradation rate may allow repeated treatment once the radionuclide is completely decayed.

The shear thinning biomaterial composition can be injected, remain intact in the physiological condition, and confine the radiation proximate to the point of injection. The radionuclide is homogenously mixed in the composition in some embodiments. The composition does not suffer precipitation or fast degradation. The presence of silicate and/or tantalum nanoparticles may augment the radiation potential as described below.

Advantages of the present disclosure include but are not limited to better therapeutic index, given localized delivery directly to the tumor target area, higher doses of radiation while limiting damage to surrounding tissues, radiosensitizing and immobilization of radionuclides, and more homogeneous distribution of radionuclides in the treatment site. The present disclosure provides a method that allows for treatment of otherwise inoperable tumors by catheter delivery, percutaneous injection, or another suitable delivery mechanism of such embolic composition.

While yttrium-90 is mentioned in this disclosure as a radionuclide, other embodiments of the present disclosure can include a variety of radionuclides including but not limited to other beta emitters such as phosphorus-32, copper 64, copper-67, iodine-131, lutetium-177, samarium-153, holmium-166, rhenium-186, and rhenium-188. The present disclosure also encompasses embodiments including alpha-emitters, including but not limited to actinium-225, bismuth-213, bismuth-212, thorium-227, radium-223, astatine-211, and terbium-149.

The present disclosure may contain an imaging agent, such as a contrast agent or speckle. Various examples of imaging materials may be utilized. The radioactive composition is a space-filling semi solid that retains radioactive material at the delivery site, preventing migration toward healthy tissues. Embodiments of the present disclosure can be used as palliative or curative treatment alone or combined with other modalities of cancer treatment. Compositions of the present disclosure may be used with contrast agent or alone or in combination with radiosensitizers to increase the potential of radiation.

### BRIEF DESCRIPTION OF THE SEVERAL VIEWS OF THE DRAWINGS

FIG. 1A shows a radioactivity signal of a radioactive shear thinning biomaterial, according to exemplary embodiments, wherein a shear thinning biomaterial is mixed with a radionuclide.

FIG. 1B shows a photograph of a syringe containing lutetium-177, as a radionuclide, after mixing with a shear thinning polymer composition, according to exemplary embodiments.

FIG. 2A shows a radioactive shear thinning biomaterial, according to exemplary embodiments, in a rabbit liver under ultrasound, wherein lutetium-177 was incorporated into a shear thinning biomaterial and delivered percutaneously to the rabbit liver under ultrasound.

FIG. 2B shows a SPECT/CT image of localized radioactivity of a radioactive shear thinning biomaterial, according to exemplary embodiments, in a rabbit liver model, wherein lutetium-177 was incorporated into a shear thinning biomaterial and delivered percutaneously to the rabbit liver under ultrasound.

FIG. 3 is a full body image of a radioactive shear thinning biomaterial, according to exemplary embodiments, in a rabbit liver under SPECT/CT. The SPECT/CT image shows the radioactivity of the shear thinning biomaterial being isolated in the area of delivery (i.e., the liver).

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Regarding microsphere treatment, yttrium-90 (⁹⁰Y) microsphere radioembolization has emerged for the management of patients with liver cancer. Two parts are present in this radioembolization procedure: embolization and brachytherapy. ⁹⁰Y is a beta emitter with a 64.2-h or 2.7 days physical half-life, in which up to 94% of the ⁹⁰Y microspheres radiation dose can be delivered during the first 11 days following treatment, after which it decays into stable zirconium. A clinical advantage of beta radiation is the ability of oncologists to prescribe and deliver relatively high doses to the tumor while minimizing dose to adjacent (nontarget) healthy tissue. ⁹⁰Y is a high-energy, β⁻-emitting radionuclide with no primary gamma emissions. The maximum energy in the ⁹⁰Y β⁻-particle spectrum is about 2.3 MeV. By Medical Internal Radiation Dose (MIRD) principle, one gigabecquerel (GBq) of ⁹⁰Y uniformly distributed throughout 1 kg of tissue provides an absorbed dose of approximately 50 Gy. Based on the cancer stage, tumor size and type the amount of delivered radiation is different.

Currently, radioembolization is indicated for the treatment of both locally advanced primary and metastatic cancers with the aim of maintaining quality of life and improving survival. While there are currently two commercially available, FDA approved ⁹⁰Y containing products used for radioembolization, such approaches, as outlined above, may elevate risks of migration out of the treatment area to non-target tissues and organs, may elevate risks of reflux into non-targeted tissues and organs, and may require delivery to the vasculature in order to be effective.

Biomaterials have been developed to allow more precise targeting of radiotherapy in order to reduce toxicity to surrounding healthy tissues and increase treatment efficacy. These unique biomaterials have been developed from polymers, glasses, and ceramics. Utilizing biomaterials for radiotherapy to deliver nanoparticles that either achieve radio sensitization of surrounding tissue producing a radiation boost or can act as radioprotectants continues to be an area of interest. The incorporation of radionuclides onto or within the structure of various biomaterials can facilitate the targeted and sustained delivery of radiotherapy to cancerous tissue. Each radionuclide has its own characteristic energy spectrum and particle emission. Biomaterials can be used to augment current methods of radiation delivery and in many instances their use can be integrated in current treatment protocols.

Of course, the main challenge of radiotherapy is that tumors are often located near normal tissues and organs at risk of radiation damage, limiting the radiation doses that can be safely delivered to the target tissue. Therefore, agents preferentially sensitizing tumors to ionizing radiation, termed radiosensitizers, have attracted great interest in radiation oncology. Tantalum-based nanoparticles can play a role in radio sensitizing or synergistic cell-killing effects for radiation therapy. Tantalum has shown a high capacity for attenuation of ionizing radiation such as X-ray, allowing it to enhance the irradiation capacity delivered directly into the tumors. Also, silicate nanoparticles may be beneficial as radiosensitizer and confinement of radioactivity to limit radiation dose outside the target tissue. Furthermore, silicate nanoparticles provide a means of immobilization of radionuclides due to the interactions with oxygen atoms of the silicate, which can enhance the ability of our shear thinning biomaterial to retain radioactivity to the treatment area.

In view of the above, the present disclosure is directed to a composition for delivery to vascularized, solid tumors via transcatheter administration, percutaneous injection, and the like.

The present disclosure relies on the ability of a shear thinning biomaterial to deliver radiation to tumor tissue or a vascular site, while being intact in the placement region with a homogenous distribution of radionuclide. For transcatheter delivery of the material, the shear thinning biomaterial also serves as an embolic agent, restricting blood flow to the tumor being treated for greater therapeutic effect. The composition may be introduced to the tumor or other lesion by means of a needle or catheter system. The composition may fill a cavity or resection site of a tumor or lesion, or it may embolize the vasculature tumors, or it may be delivered directly to the solid tissue. The slow resorption over the half-life or whole shelf life of the radioisotope, allows for continued delivery of radioactivity. The present disclosure allows incorporation of chemotherapy/immune therapy drugs, sensitizing materials, and different methods of delivery, while preventing the leaching of the radioactive source to the non-target tissues.

The present disclosure provides a method of internal radiotherapy combining a high energy radiation source combined with shear thinning biomaterial with a preferred viscosity, thereby delivering radiation preferentially via catheter, percutaneous injection, and/or other suitable delivery route. Incorporation of radiation, through either a high energy beta or alpha emitter, concentrates the zone of radiation exposure to the vicinity of the tumor and reduces the radiation level and risk of damage to surrounding healthy tissue. The slow resorption of the composition generously exceeds the half-life of various radionuclides of interest which advantageously reduces the risk of leaking to the other organs and tissues. The slow degradation may allow repeated treatment once the radionuclide is completely decayed.

As indicated, the shear thinning biomaterial composition of the present disclosure can be injected and remain intact in the physiological condition and confine the radiation at the point of injection. The radionuclide can be homogenously mixed in the composition. The composition does not precipitate or degradation prematurely. The presence of silicate and/or tantalum nanoparticles may augment the radiation potential. Advantages of the enclosed invention include but are not limited to better therapeutic index given localized delivery directly to the tumor target area, higher doses of radiation while limiting damage to surrounding tissues, radiosensitization and immobilization of radionuclides, and more homogeneous distribution of radionuclides in the treatment site.

In an embodiment, a relative position of the radioactive material within the shear thinning biomaterial composition, after mixing with the shear thinning biomaterial, remains unchanged before, during, and after flowing (e.g. injection) of the shear thinning biomaterial. In other words, a homogenous distribution of radioactive material within the shear thinning biomaterial composition is present before and after implantation and or during injection to the treatment site.

According to an embodiment, a composition of the present disclosure can be modulated by: (1) varying radionuclides for transmission of radiotherapy—this includes alpha or beta emitting radionuclides; (2) controlling viscosity to modulate diffusion into the tissue for percutaneous delivery and permit the radionuclides to concentrate and remain in the tumor; and (3) incorporating other drugs (e.g., chemotherapeutics and immune-therapy compounds) and releasing them within target tissue (e.g., tumors) in a controlled manner.

In an embodiment, the shear thinning composition disclosed herein can be optimized for therapeutic ratio by (1) employing a high-energy, pure beta-emitter (such as ⁹⁰Y), (2) confining the radioactive source to the tumor, (3) distributing the beta-emitter as uniformly as possible within the tumor, and (4) acting as a radiosensitizer, thereby increasing therapeutic effect. The injectable composition disclosed herein can be used as a carrier to increase the retention of radionuclide in the tumors and reduce leakage and systematic toxicity. The composition herein has shear thinning properties, flowing readily for injection while staying in place upon extrusion in the tumor. Additionally, the composition can be loaded with any other type of radioactive material.

Compositions according to the present disclosure are highly stable to radiation and incorporation of radioisotopes may augment current methods of radiation delivery. Further, the composition can incorporate chemotherapy or immunotherapy drug.

According to an embodiment, the present disclosure relates to a method of preparing a radioactive shear thinning biomaterial, comprising the steps of 1) fabricating a biomaterial and 2) mixing the resulting composition with a radioisotope. Accordingly, compositions of the present disclosure may comprise a biocompatible polymer, a synthetic silicate nanoparticle, a biocompatible solvent, and from about 0.1 weight percent to about 25 weight percent of a radioisotope having radioactive content of from about 0.5 microcurie to about 100 millicurie. In an embodiment, the composition may further comprise a non-radioactive contrast agent.

The biomaterial of the composition may be a mixture of the biocompatible polymer, the synthetic silicate nanoparticle, and the biocompatible solvent. The mixture may include, for example, a range of silicate nanoparticles with concentrations between 0.1% to 50%, a range of biocompatible polymer with concentrations between 0.5% to 20%, and the solvent as the balance. Unless otherwise indicated, percentages (%) expressed herein are weight percentages. The mixture may include silicate nanoparticles in an amount ranging anywhere from 0.1% to 0.2% to 0.5% to 1% to 2% to 5% to 10% to 15% to 20% to 30% to 40% to 50% (in other words, ranging between any two of the preceding values). The mixture may include, for example, biocompatible polymer an amount ranging anywhere from 0.5% to 1% to 2% to 5% to 10% to 15% to 20%.

The radioactive shear thinning biomaterial may comprise a mixture of the biocompatible polymer, the synthetic silicate nanoparticle, the radionuclide, and the biocompatible solvent. The radioactive shear thinning biomaterial may include, for example, a range of silicate nanoparticles with concentrations between 0.1% to 50%, a range of biocompatible polymer with concentrations between 0.5% to 20%, a range of radionuclide with concentrations between 0.1% and 40%, and the solvent as the balance. The radioactive shear thinning biomaterial may include silicate nanoparticles in an amount ranging anywhere from 0.1% to 0.2% to 0.5% to 1% to 2% to 5% to 10% to 15% to 20% to 30% to 40% to 50%. The radioactive shear thinning biomaterial may include, for example, biocompatible polymer an amount ranging anywhere from 0.5% to 1% to 2% to 5% to 10% to 15% to 20%. The radioactive shear thinning biomaterial may include, for example, radionuclide an amount ranging anywhere from 0.1% to 0.2% to 0.5% to 1% to 2% to 5% to 10% to 15% to 20% to 30% to 40%.

In a preferred embodiment, the amount and radioactive content of the radioisotope is sufficient to provide for a cumulative ionizing radiation dosage at the site of implantation from about 200 to about 100,000 rads [2-1000 Gray (Gy)].

In the preferred embodiment, compositions described herein are employed to effect necrosis of at least a portion of solid tumor. Accordingly, the compositions are delivered, for example, directly to the solid tumor or to a vascular site selected to be in or near the solid mass tumor, and the amount and radioactive content of the radioisotope employed in the composition is sufficient to effect such necrosis.

In an embodiment, a method of the present invention for making a radioisotope composition includes mixing shear thinning biomaterial with an aqueous non soluble or confined radioisotope. In one embodiment, an already activated radioisotope is incorporated into the biomaterial for administration based on desired dose. In another embodiment, a radionuclide precursor is incorporated in the biomaterial and subsequently activated through neutron bombardment. In another embodiment, the radioactive content is a naturally emitting radioactive content.

The term radioisotope refers to naturally or non-naturally occurring radioisotopes conventionally used in nuclear medicine including, by way of example, only, ⁹⁰yttrium, ¹⁹²iridium, ¹⁹⁸gold, ¹²⁵iodine, ¹³⁷cesium, ⁶⁰cobalt, ³²phosphorous, ⁵²magnesium, ⁵⁵iron, ⁹⁰strontinum, different cobalt. Other radionuclides currently being produced for use in nuclear medicine include for example, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ²⁰³lead, ¹¹¹indium, ⁵²iron, ¹⁶⁷thulium, ⁵⁷nickle, ⁶²zinc, ⁶¹copper, ¹²³iodine.

The biocompatible polymer employed in these compositions and methods can be either a biodegradable polymer or a non-biodegradable polymer but is preferably biodegradable. Biodegradable polymers are disclosed in the art. For example, linear chain polymers such as gelatin, collagen, protein, alginate, agar, polysaccharide, chitosan, polyvinyl alcohol, polylactide, polyglycolides, polycaprolactones, polyanhydrides, polyamides, polyurethanes, polyethylene glycol, and copolymers, terpolymers and combinations thereof.

The term contrast agent refers to a biocompatible radiopaque material capable of being monitored during injection, for example, radiography. Example of contrast agents include Tantalum, tantalum oxide, gold, tungsten, platinum powder, barium sulphate, and Omnipaque™ (iohexol).

In one embodiment, the radioisotope acts as a contrast agent to permit visualization of the composition during catheter delivery. Alternatively, a non-radioactive contrast agent is employed in combination with the radioisotope to ensure visualization.

In one embodiment, radioisotopes having a sufficiently high atomic number so as to be radiopaque can be used to serve both as a source of radiation and contrast agent for detection under fluoroscopy.

In another embodiment, a separate non-radioactive contrast agent is employed in conjunction with the radioisotope.

According to an embodiment, the radioactive composition is a shear-thinning composition. Shear thinning is a non-Newtonian behavior of fluids whose viscosity decreases under strain. In other words, as certain forces (i.e., shear) are applied to such shear thinning fluids, the fluids more readily flow. This allows the shear-thinning composition to be more easily delivered via catheter, percutaneously, and the like.

Moreover, the composition may have mechanical properties similar to that of tissue proximate the composition upon implantation. For instance, a storage modulus (G′) of the composition may be between 1 kPa to 1 MPa. In embodiments, the storage modulus (G′) of the composition may be between 1 kPa and 100 kPa. In embodiments, the storage modulus (G′) of the composition is between 1 kPa and 40 kPa. As can be appreciated, the mechanical properties of the composition are dictated, in part, by the anticipated mechanical properties of tissues expected to be proximate the implanted composition.

In embodiments, the yield stress of the composition is from about 1 Pa to about 200 Pa. In some embodiments, the yield stress of the composition is from about 1 Pa to about 100 Pa. In embodiments, the yield stress of the composition is from about 2 Pa to about 50 Pa. In embodiments, the yield stress of the composition is from about 1 Pa to about 25 Pa. In embodiments, the yield stress of the composition is from about 1 Pa to about 10 Pa. In embodiments, the yield stress of the composition is from about 1 Pa to about 5 Pa. In embodiments, the composition flows upon application of a pressure greater than the yield stress.

In an embodiment, the phase transitioning qualities of the composition are determined by, among other things, ratios of ingredients within the composition and/or total solid content of the composition. The ratios of ingredients (e.g., ratios of oppositely charged polymers and nanoparticles) can impact electrostatic interactions. Together, the ratios of ingredients and total solid content determine viscoelastic properties (e.g., how the viscosity changes under shear rate and the extent of recovery/reversibility) of the composition.

In an embodiment, wherein the composition is integrated with a chemotherapeutic or other drug or biologic treatment, the preferred composition maybe mixed with the therapy. For example, the composition can be mixed with Doxorubicin.

According to an embodiment, compositions described above can be employed in the treatment of solid tumors. In one such treatment, these compositions are employed in methods for needle or catheter assisted embolization of blood vessels. The injection of the shear thinning composition maybe performed intraoperatively or percutaneously. In such methods, an amount of the composition is introduced into the selected vessel via a needle or catheter delivery under fluoroscopy so that the blood vessel is embolized in the case of catheter delivery. In other embodiments, the composition is injected directly into the solid tumor.

According to an embodiment, the compositions described herein are useful in the necrosis of solid tumor by, for example, embolization of blood vessels leading to or within the solid mass tumor. When employed to embolize blood vessels, it is preferred that the level of radiation employed in the composition is sufficient to also ablate at least portion of tumor. Alternatively, the composition can be delivered directly into the solid tumor mass and the radiation contained therein can be employed to effect necrosis of tumor.

It is contemplated that the compositions described herein can be employed as a carrier for a chemotherapeutic or immunotherapy agents wherein the agent is delivered for subsequent release to the solid tumor.

In an embodiment of the present disclosure, the shear thinning biomaterial is suitable for syringe injection through a needle allowing the hydrogel to infiltrate the tumor site to deliver the radioactive dose, thereby permitting percutaneous delivery of radionuclides to the tumor site. The high viscosity of the compositions described herein ensures homogeneity of the delivered radionuclides throughout the tumor tissue while keeping the radioactive source confined to the site of delivery. This minimizes the chance of radiation impacting surrounding, healthy tissues via radionuclide migration.

### EXAMPLES

### Example 1

The purpose of this example is to demonstrate the preparation of a composition in accordance with this invention. The composition comprises (a) Gelatin, (b) Silicate nanoparticle, and (c) Water. After mixing of all ingredients, this composition was then added to contrast agent and the resulting composition was mixed thoroughly by speed mixer followed by curing. The cured composition was mixed with the radioisotope.

### Example 2

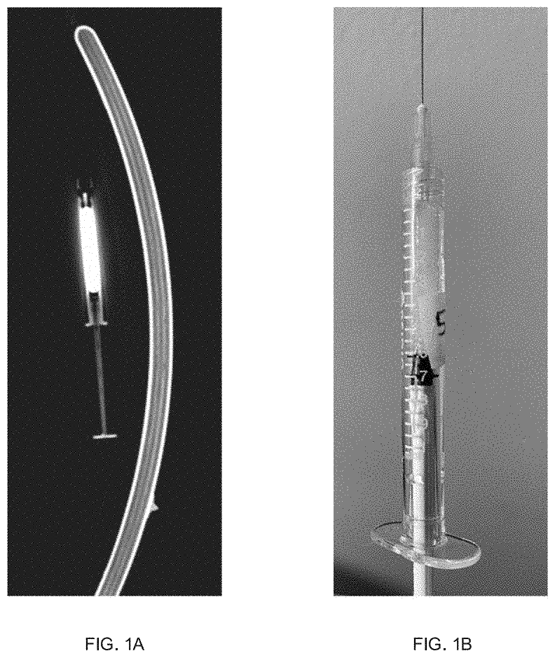

The purpose of this example is to demonstrate the preparation of a composition in accordance with this invention. The composition comprises (a) Gelatin, (b) Silicate nanoparticle, and (c) Water. After mixing of all ingredients, this composition was cured. The cured composition was mixed with radioisotope, i.e., lutetium-177. Example 2 is shown in FIG. 1A through FIG. 3.

For instance, FIG. 1A and FIG. 1B show an example of mixing a lutetium-177 radionuclide with a biocompatible polymer of the present disclosure as an injectable solid. FIG. 1A shows the radioactivity signal of radioactive shear thinning biomaterial. FIG. 1B shows a photograph of the syringe containing lutetium-177 after mixing.

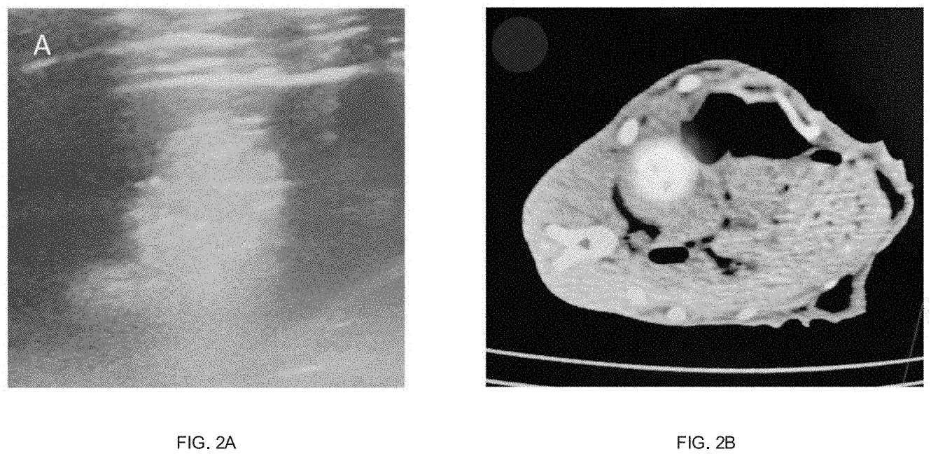

FIG. 2A and FIG. 2B show the radioactive, shear thinning composition in a rabbit liver under ultrasound (FIG. 2A) or SPECT/CT (FIG. 2B). For this figure, lutetium-177 was incorporated into the shear thinning biomaterial and delivered percutaneously to the liver under ultrasound.

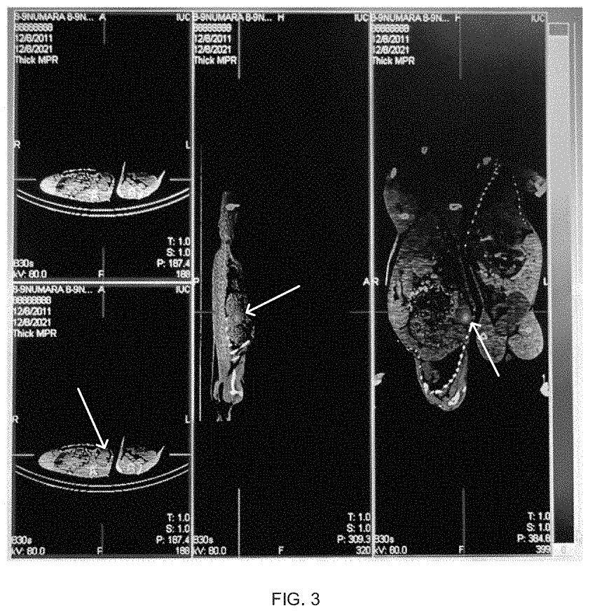

FIG. 3 shows the radioactive, shear thinning composition in a rabbit liver under SPECT imaging in context of the full rabbit. The SPECT/CT imaging shows the radioactivity is isolated in the area of delivery (i.e., the liver) and does not travel away from the treatment site.

### Example 3

The purpose of this example is to demonstrate the preparation of a composition in accordance with this invention. The composition comprises (a) Gelatin, (b) Silicate nanoparticle, and (c) Water. After mixing of all ingredients, this composition will be mixed with non-radioactive seed and the mixture will be bombarded and activated (e.g., through neutron bombardment) and radioisotope becomes activated.

1. A composition suitable for treating a solid tumor in which the composition comprises:
a biocompatible polymer;
silicate nanoparticles;
water; and
a radionuclide.

2. The composition of claim 1, wherein said biocompatible polymer is selected from the group consisting of gelatin, collagen, alginate, silk, agar, polysaccharide, cellulose, hydroxpropylmethyl cellulose, chitosan, polyvinyl alcohol, polylactide, polyglycolide, polycaprolactone, polyanhydride, polyamide, polyurethane, polyethylene glycol (PEG), polyhydroxycellulose, polytetrafluoroethylene (PTFE), polylactic acid (PLA), poly-(1)-lactic acid (PLLA), poly (d)-lactic acid (PLDA), agarose, starch, lignin, keratin, polyvinyl alcohol (PVA), and copolymers, terpolymers and combinations thereof.

3. The composition of claim 1, wherein the composition comprises about 0.5% to about 20% (w/w) of one or more of the biocompatible polymers.

4. The composition of claim 1, wherein the silicate nanoparticles is selected from the groups consisting of synthetic silicate nanoparticles (laponite) and natural silicate nanoparticles (phyllosilicate, bentonite, kaolinite, montmorillonite-smectite).

5. The composition of claim 1, further comprising a contrast agent.

6. The composition of claim 5, wherein the contrast agent is present in an amount of from about 10 to about 40 weight percent of contrast agent.

7. The composition of claim 5, wherein the contrast agent is selected from the group consisting of tantalum, tungsten, platinum, gold, and iohexol.

8. The composition of claim 1, wherein the compositions comprises the radionuclide in an amount of from about 0.1 to 40 weight percent.

9. The composition according to claim 1, wherein the radioactive dosage is tunable for the specific tumor and patient requirements as determined by physicians.

10. The composition of claim 1, wherein said radionuclide is selected from a group of radionuclides including ⁹⁰Y, ¹⁷⁷Lu, ³²P, ¹⁹⁸Au, ¹²⁵I, ¹³¹I, ⁶⁰Co, ¹³⁷Ce, and ¹⁶⁶Ho.

11. The composition of claim 1, wherein the silicate nanoparticles serve as radiosensitizers for the composition.

12. A composition of claim 1, comprising a plurality of differing radionuclides.

13. A method of treating a solid tumor comprising delivering a composition via catheter intravascularly to the solid tumor, wherein the composition comprises:
a biocompatible polymer;
silicate nanoparticles;
water; and
a radionuclide.

14. The method of claim 13, wherein the composition both embolizes a blood vessel supplying blood to the tumor and causes necrosis of the tumor.

15. The method of claim 13, wherein said biocompatible polymer is selected from the group consisting of gelatin, collagen, alginate, silk, agar, polysaccharide, cellulose, hydroxpropylmethyl cellulose, chitosan, polyvinyl alcohol, polylactide, polyglycolide, polycaprolactone, polyanhydride, polyamide, polyurethane, polyethylene glycol (PEG), polyhydroxycellulose, polytetrafluoroethylene (PTFE), polylactic acid (PLA), poly-(l)-lactic acid (PLLA), poly (d)-lactic acid (PLDA), agarose, starch, lignin, keratin, polyvinyl alcohol (PVA), and copolymers, terpolymers and combinations thereof.

16. The method of claim 13, wherein the silicate nanoparticles are selected from the groups consisting of synthetic silicate nanoparticles (laponite) and natural silicate nanoparticles (phyllosilicate, bentonite, kaolinite, montmorillonite-smectite).

17. The method of claim 13, wherein said radionuclide is selected from a group of radionuclides including ⁹⁰Y, ¹⁷⁷Lu, ³²P, ¹⁹⁸Au, ¹²⁵I, ¹³¹I, ⁶⁰Co, ¹³⁷Ce, and ¹⁶⁶Ho.

18. A method of treatment comprising delivering a composition percutaneously directly to a tumor site or filling residual space in a surgically debulked tumor, wherein the composition comprises:
a biocompatible polymer;
silicate nanoparticles;
water; and
a radionuclide.

19. The method of claim 18, wherein said biocompatible polymer is selected from the group consisting of gelatin, collagen, alginate, silk, agar, polysaccharide, cellulose, hydroxpropylmethyl cellulose, chitosan, polyvinyl alcohol, polylactide, polyglycolide, polycaprolactone, polyanhydride, polyamide, polyurethane, polyethylene glycol (PEG), polyhydroxycellulose, polytetrafluoroethylene (PTFE), polylactic acid (PLA), poly-(l)-lactic acid (PLLA), poly (d)-lactic acid (PLDA), agarose, starch, lignin, keratin, polyvinyl alcohol (PVA), and copolymers, terpolymers and combinations thereof and/or wherein the silicate nanoparticles are selected from the groups consisting of synthetic silicate nanoparticles (laponite) and natural silicate nanoparticles (phyllosilicate, bentonite, kaolinite, montmorillonite-smectite).

20. The method of claim 18, wherein said radionuclide is selected from a group of radionuclides including ⁹⁰Y, ¹⁷⁷Lu, ³²P, ¹⁹⁸Au, ¹²⁵I, ¹³¹I, ⁶⁰Co, ¹³⁷Ce, and ¹⁶⁶Ho.
